(19) United States
(12) Patent Application Publication
(10) Pub. No.: US 2013/0310770 A1
(43) Pub. Date: Nov. 21, 2013

(54) Infusion Apparatus With Composition Pulse Flow Sensor

(71) Applicants: Dominic J. Cooke, Mission Viejo, CA (US); Siddharth Desai, Ladera Ranch, CA (US)

(72) Inventors: Dominic J. Cooke, Mission Viejo, CA (US); Siddharth Desai, Ladera Ranch, CA (US)

(21) Appl. No.: 13/474,445

(22) Filed: May 17, 2012

(51) Int. Cl. A61M 5/00

(52) U.S. Cl. 604/257

(57) ABSTRACT

A device for dispensing fluid to a patient and indicating a fluid flow condition. The device includes a reservoir configured to provide a source of fluid under pressure. A continuous flow path in fluid communication with the source of fluid provides a continuous and substantially constant flow rate of fluid from the source to a patient. The device further includes at least one composition pulse flow sensor in fluid communication with the continuous flow path. The composition pulse flow sensor is configured to provide a signal that the flow rate of the fluid in the continuous flow path is different from a predetermined flow rate, thereby indicating a fluid flow condition.

### FIELD OF THE INVENTION

This invention relates to liquid dispensing systems, and more specifically to a catheter-based system for infusing a liquid into the body of a patient, and most specifically to a pain management system which administers a post-operative drug to a wound site of a patient through a catheter that delivers fluid medication uniformly and at a known rate across an infusion section of the catheter.

### BACKGROUND

In instances of severe pain, infection, and other medical ailments, it has been proven beneficial to administer a continuous flow of medicinal fluid to a patient through a catheter-based system. There are many types of medicinal fluids that can be administered in this manner including, but not limited to, insulin, analgesics and antibiotics.

The continuous delivery of such medicinal fluids over extended periods of time has required prolonged hospital stays and monitoring by medical staff. Devices for this purpose have been designed to be fairly mobile and provide for a continuous or basal rate of fluid, which is the on-going continuous primary flow rate of fluid to a patient.

However, one problem that is not successfully addressed is readily determining whether the flow of fluid to the patient has been altered or interrupted. Very often, the rates of flow are in the range of from about 0.5 to about 14 cubic centimeters of fluid per hour. At such low flow rates, it is difficult to determine if the flow is inadvertently altered or interrupted by, for example, material collecting in a filter, orifice, connection, or in a flow regulator to block or alter the flow rate. Alternatively and/or additionally, the flow path may become pinched, constricted or kinked to alter or interrupt the flow rate. An interruption in flow alters the pressure of fluid in the tubing.

Various hydrostatic manometers have been developed that may be directly placed in the tubing line and that may be operated to temporarily interrupt the fluid flow so that hydrostatic pressure measurements may be periodically taken. See, for example, U.S. Pat. No. 3,807,389 to Miller et al. These types of in-line manometers measure hydrostatic pressure and require periodic interruption of the fluid flow, such as by a stopcock, to obtain a pressure reading. This is inconvenient in some situations and may even be hazardous if the required pressure level drops or rises significantly between readings, resulting in over-or under infusion.

An in-line, hydrodynamic manometer for measuring infusion pressures is described in U.S. Pat. No. 4,282,881 to Todd et al. This manometer uses a closed pressure-measuring chamber containing a nonexpansible volume of air, which is in communication with a passage through which fluid, whose pressure is to be measured, flows. Several problems exist with this manometer design. For example, the entire apparatus is rather large in order to accommodate a pressure-measuring chamber long enough to measure a given range of pressures. The manometer, as illustrated in FIG. 1 of U.S. Pat. No. 4,282,881, is large enough to require support on a stand.

There are numerous markings on the housing of the manometer, as shown in FIG. 2 of U.S. Pat. No. 4,282,881, which correspond to various hydrodynamic pressure readings of the fluid flowing through the passage. Again, this results in the need for a relatively long pressure-measuring chamber and thus a relatively large manometer apparatus. Furthermore, because the pressure of intravenous infusions is typically low, from approximately 6 psi at the fluid source to approximately 0.3 psi at the patient's vein, clinical personnel generally do not care about, nor do they need to know, absolute hydrodynamic pressures during intravenous (“IV”) infusion of fluid.

What is clinically important is whether and when the flow is in one of three states: 1) flowing relatively freely; 2) obstructed by a distal blockage (i.e., downstream from the manometer, typically at the site of insertion of the catheter into the patient); or 3) not flowing at all, either because the infusion is turned off or there is a proximal obstruction (i.e., upstream from the manometer, typically close to the fluid source and/or within the associated delivery tubing). Thus, the traditional manometer scale with a wide array of absolute pressure markings is, generally, clinically unnecessary.

An improved manometer is described in U.S. Pat. No. 6,371,937. This device functions as a conventional manometer with a pressure-measuring chamber but includes an additional space-saving chamber connected to the pressure-measuring chamber that allows the manometer to be much smaller than conventional devices. Fluid flows through the device and also enters the pressure-measuring chamber where it reaches a level through compression and expansion of air in both the pressure-measuring chamber and space-saving chamber. This scaled down device includes simple markings corresponding to fluid flow states. However, the device is still a manometer and required fluid to enter a pressure-measuring chamber. Moreover, the device must be aligned and oriented properly to obtain a reading. That is, the flow state of fluid within the passage is determined by an examiner, typically a nurse or other caregiver, by ascertaining where the leading edge, or top, of the fluid column within the pressure-measuring chamber is in comparison to certain reference markings that are associated with, and are present alongside, the pressure-measuring chamber. In addition to these problems, at very low flow rates and/or very low pressures (e.g., essentially atmospheric pressures) changes in the flow rate or pressure are difficult to detect.

What is needed is a simple, mobile device to provide a continuous and substantially constant flow of medicinal fluid and indicate a fluid flow condition in a clear, discrete and easy to identify manner. Further, a simple and effective device that indicates a fluid flow condition in a clear, discrete and easy to identify manner such that it can be readily identified by even a busy care provider or an infirm patient.

Accordingly, there is a need for an indicator assembly that can be readily integrated into liquid dispensing systems, and more specifically to a catheter-based system for infusing a liquid into the body of a patient and which is easy to view and read properly and function at low flow rates of less than 14 cubic centimeters of fluid per hour, desirably between 0.5 and 14 cubic centimeters per hour. There is also a need for an indicator assembly that can be readily integrated into a catheter-based liquid dispensing system for infusing a liquid into the body of a patient and which is easy to view and read properly and function at relatively low flow rates and at pressures less than about 4 pounds per square inch (28 kilopascals).

A need exists for an indicator assembly that be readily integrated into a catheter-based liquid dispensing system for infusing a liquid into the body of a patient that is simple, reliable and accurate. A need also exists for an indicator assembly that be readily integrated into a catheter-based liquid dispensing system for infusing a liquid into the body of a patient that is simple, reliable and accurate at indicating predetermined pressures as well as easy to understand. There is also an unmet need for a pressure change indicator assembly that conveys a simple and easy to see and understand signal about a change in a fluid flow condition.

### SUMMARY OF THE INVENTION

In response to the difficulties and problems discussed herein, the present invention provides a device for dispensing fluid to a patient and indicating a fluid flow condition. The device includes a reservoir configured to provide a source of fluid under pressure. A continuous flow path in fluid communication with the source of fluid provides a continuous and substantially constant flow rate of fluid from the source to a patient. The device further includes at least one composition pulse flow sensor in fluid communication with the continuous flow path. The composition pulse flow sensor is configured to indicate a fluid flow condition.

In an aspect of the invention, the continuous flow path may include a flow regulator which sets the flow rate through the continuous flow path into the patient. The composition pulse flow sensor may be located between the flow regulator and the source of fluid. Alternatively and/or additionally, the composition pulse flow sensor may be located in a direction downstream of the flow regulator.

The composition pulse flow sensor is configured to indicate a fluid flow state. For example, the composition pulse flow sensor may be configured to provide a discrete visual signal, an audible signal, and/or a tactile signal. Alternatively and/or additionally, the composition pulse flow sensor may be configured to provide an electronic signal to another device such as a display, an alarm, and/or a device such as a phone or other communication device. The signal and/or display can indicate a flow state that is different from a predetermined flow state. Alternatively and/or additionally, the signal and/or display can indicate a flow state that is a continuous and substantially constant flow rate of fluid. In yet another aspect of the invention, the signal and/or display can indicate a flow state that is less than a continuous and substantially constant flow rate of fluid.

In another aspect of the invention, when a composition pulse flow sensor is located between a flow regulator (or other potential source of obstruction such as a filter or bubble-trap) and the pressurized source of fluid and the composition pulse flow sensor provides information of the flow condition of the fluid in the continuous flow path (e.g., that flow state that is greater than, equal to, and/or less than a continuous and substantially constant flow rate of fluid). In yet another aspect of the invention, when a composition pulse flow sensor is located in a direction downstream of a flow regulator (or other potential source of obstruction such as a filter or bubble-trap) and the composition pulse flow sensor provides information of the flow condition of the fluid in the continuous flow path e.g., that flow state that is greater than, equal to, and/or less than a continuous and substantially constant flow rate of fluid).

Generally speaking, the composition pulse flow sensor includes a housing, a channel in the housing for a fluid to be measured, a semiconductor chip arranged in the housing at the channel, the semiconductor chip having an integrated flow sensor. The housing will typically define an inlet for receiving fluid from the continuous flow path and an outlet for returning fluid to the continuous flow path. For example, the housing of the composition pulse flow sensor may include a first end, a second end, one or more walls defining an interior channel, and an axial dimension. At least the first end of the housing is in fluid communication with the continuous flow path. The integrated flow sensor of the composition pulse flow sensor may be a composition pulse time-of-flight flow sensor composed of one or more electrodes that generate a compositional variation in a laminar liquid flow and at least one sensor (e.g., a conductivity sensor) the variation. The integrated flow sensor is desirably configured to determine flow velocity or the mass flow of the fluid in the continuous flow path.

According to an aspect of the invention, the composition pulse flow sensor includes a channel substrate; at least one channel arranged in the channel substrate for receiving the fluid, wherein the channel is formed by at least one recess in the channel substrate; a sensor substrate arranged at the channel substrate; and a composition pulse flow sensor arranged on the sensor substrate, the flow sensor comprising at least one electrode and at least one sensor integrated on the sensor substrate. The channel substrate includes a cover layer covering and closing the recess in the channel substrate. The electrode and the sensor are in fluid contact with the cover layer and thereby with the channel, and the sensor substrate is arranged on an outer side of the cover layer.

The present invention also encompasses an indicator assembly for indicating a fluid flow state in a medical device for dispensing a fluid under pressure to a patient through a continuous flow path at a continuous and substantially constant flow rate of fluid. The indicator assembly includes a continuous flow path from a source providing a continuous and substantially constant flow rate of fluid and a composition pulse flow sensor in fluid communication with the continuous flow path. The composition pulse flow sensor provides a first signal when the flow rate of fluid in the flow path is at a predetermined flow rate and a second signal when the flow rate of fluid in the flow path is no longer at a predetermined flow rate, such that the second discrete visual signal provides warning that indicates a flow state that is less than a continuous and substantially constant flow rate of fluid. The composition pulse flow sensor may be configured to be a binary indicator and provide no signal of other pressure states between a first discrete visual signal and a second discrete visual signal. Alternatively and/or additionally, the composition pulse flow sensor can be configured with a signal and/or display that indicate a flow state that is different from a predetermined flow state (e.g., that provides information about a flow rate in terms of mass, volume, or flow velocity). Alternatively and/or additionally, the signal and/or display can indicate a flow state that is a continuous and substantially constant flow rate of fluid. In yet another aspect of the invention, the signal and/or display can indicate a flow state that is less than a continuous and substantially constant flow rate of fluid. The indicator may provide a discrete visual signal, an audible signal, and/or a tactile signal. Alternatively and/or additionally, the composition pulse flow sensor may be configured to provide an electronic signal to another device such as a display, an alarm, and/or a device such as a phone or other communication device.

Generally speaking, the composition pulse flow sensor is as described above. For example, the composition pulse flow sensor may include a housing, a channel in the housing for a fluid to be measured, a semiconductor chip arranged in the housing at the channel, the semiconductor chip having an integrated flow sensor. The housing may define an inlet for receiving fluid from the continuous flow path and an outlet for returning fluid to the continuous flow path. For example, the housing of the composition pulse flow sensor may include a first end, a second end, one or more walls defining an interior channel, and an axial dimension. At least the first end of the housing is in fluid communication with the continuous flow path. The integrated flow sensor of the flow detector is a composition pulse time-of-flight flow sensor composed of one or more electrodes that generate a compositional variation in a laminar liquid flow and at least one sensor (e.g., a conductivity sensor). The integrated flow sensor is configured to determine flow velocity or the mass flow of the fluid in the continuous flow path.

The present invention encompasses a system for dispensing fluid to a patient and indicating a fluid flow condition. The system includes: a reservoir for providing a source of fluid under pressure; a continuous flow path in fluid communication with the source of fluid for providing a continuous and substantially constant flow rate of fluid from the source; and at least one composition pulse flow sensor in fluid communication with the continuous flow path, such that the at least one composition pulse flow sensor provides a signal when the flow rate of the fluid in the continuous flow path is different from a predetermined flow rate, thereby indicating a fluid flow condition (e.g., a fluid flow rate).

The system may further include a flow regulator and a composition pulse flow sensor may be located between the flow regulator and the source of fluid such that the composition pulse flow sensor provides a signal when the flow rate of the fluid in the continuous flow path is less than the predetermined flow rate and such a signal indicates a flow state that is less than a continuous and substantially constant flow rate of fluid. Alternatively and/or additionally, the system may include a flow regulator and a composition pulse flow sensor may be located in a direction downstream of a flow regulator such that the composition pulse flow sensor provides a signal when the flow rate of the fluid in the continuous flow path is less than the predetermined flow rate and such a signal indicates a flow state that is less than a continuous and substantially constant flow rate of fluid.

A better understanding of the above and many other features and advantages of the liquid dispensing device with flow indicator may be obtained from a consideration of the detailed description of the invention below, particularly if such consideration is made in conjunction with the appended drawings.

### BRIEF DESCRIPTION OF THE DRAWINGS

FIG. 1 is a schematic view of an embodiment of the present invention illustrating an exemplary device for dispensing fluid to a patient which includes a flow indicator

FIG. 2 is a schematic view of an embodiment of the present invention illustrating an exemplary device for dispensing fluid to a patient which includes a flow indicator as well as a large volume bolus delivery system.

FIG. 3A is a side view of a portion of an exemplary device for dispensing fluid to a patient incorporating a composition pulse flow sensor assembly.

FIG. 3B is a perspective view showing a detail of a feature from a portion of an exemplary composition pulse flow sensor incorporated in a device for dispensing fluid to a patient.

FIG. 3C is a side view showing a cross-section of a portion of an exemplary composition pulse flow sensor incorporated in a device for dispensing fluid to a patient.

### DETAILED DESCRIPTION OF THE INVENTION

The present invention relates generally to liquid dispensing systems, and more specifically to a catheter-based system for infusing a liquid into the body of a patient. More particularly, the invention relates to a pain management system which administers a post-operative drug to a wound site of a patient through a catheter that delivers fluid medication uniformly and at a known rate across an infusion section of the catheter and which incorporate a composition pulse flow sensor that provides a signal a flow condition in the continuous flow path is different from a predetermined flow condition. The invention disclosed herein also relates to a composition pulse flow sensor assembly for use with a fluid delivery device in which the composition pulse flow sensor assembly includes a composition pulse flow sensor that provides a signal that a flow condition in a continuous flow path of such a fluid delivery device is different from a predetermined flow condition.

Generally speaking, the flow detectors utilized in the present invention may be described as micro-machined composition pulse “time-of-flight” mass flow sensors that utilize general principles of the “time-of-flight” of a localized compositional variation that is generated in a liquid by electrolysis of the fluid or by electrodialysis. One or more electrodes, which may be in the form of a wire or thin film immersed in a fluid flow, can be used to generate the localized compositional variation which is then detected by sensor (e.g., a conductivity sensor) and used to measure the mass velocity at a point in the flow.

Useful flow detectors for the present invention are described at, for example, U.S. Pat. No. 6,675,660 granted Jan. 13, 2004 to Mosier et al. for “Composition Pulse Time-of-Flight Mass Flow Sensor”; U.S. Pat. No. 7,225,683 granted Jun. 5, 2007 to Harnett et al. for “Composition Pulse Time-of-Flight Mass flow Sensor”; and U.S. Pat. No. 7,703,336 granted Apr. 27, 2010 to Genosar for “Multi-Sensor Mass Flow Meter Along with Method for Accomplishing Same”; the entire contents of each of these patents are incorporated herein by reference.

The present invention utilizes such composition pulse flow sensors for liquid flow applications. More particularly, the present utilizes such composition pulse flow sensors for laminar liquid flow applications. Unlike thermal mass flow meters that use heat to measure flow, the composition pulse flow sensors generates a localized variation in the composition of the fluid flow (desirably laminar fluid flow). The composition pulse flow sensors introduce a compositional variation into the flow stream utilizing electrodes and a sensor measures the compositional variation at a location downstream of the electrodes. The present invention avoids many detection problems by utilizing the flow detectors with relatively low flow rates of liquid under laminar flow conditions and by pulsed operation of the electrodes.

The invention operates by producing localized compositional variations in the fluid at distinct locations along the flow axis. The time required for the compositional variation, or pulse, to be detected downstream from its point of creation is used to derive a flow rate. The pulse, comprising a narrow zone in the fluid whose composition is different from the mean composition of the fluid, can be created by electrochemical means, such as by electrolysis of a solvent, electrolysis of a dissolved species, or electrodialysis of a dissolved ionic species.

According to an aspect of the invention, relatively dilute solutions of active ingredients in an inert carrier such as, for example, sodium chloride solution are desirably used as the fluid. Ingredients such as medicines, anesthetics, antibiotics, or the like at dilute concentrations (1% or less) are desirably used in an inert carrier that can briefly dissociate or temporarily change composition when subject to energy input from an electrode or thin film. It is contemplated in some aspects of the invention, that at a sufficiently low flow rate of the fluid, the dissociation or composition change may register on an adjacent sensor but be substantially or completely reversed before the fluid reaches a patient. Alternatively and/or additionally, at very low flow rates, the amount of dissociation or composition change required to register on an sensor adjacent an electrode or thin film would be very low such that any decomposition products or other compounds generated would be minimal or negligible. Exemplary fluids may be, for example, bupivacaine hydrochloride solution 0.25% w/v & 0.5% w/v (equivalent toanhydrous bupivacaine hydrochloride 2.5 mg/ml and 5 mg/ml).

Reference will now be made in detail to one or more embodiments of the invention, examples of the invention, examples of which are illustrated in the drawings. Each example and embodiment is provided by way of explanation of the invention, and is not meant as a limitation of the invention. For example, features illustrated or described as part of one embodiment may be used with another embodiment to yield still a further embodiment. It is intended that the invention include these and other modifications and variations as coming within the scope and spirit of the invention.

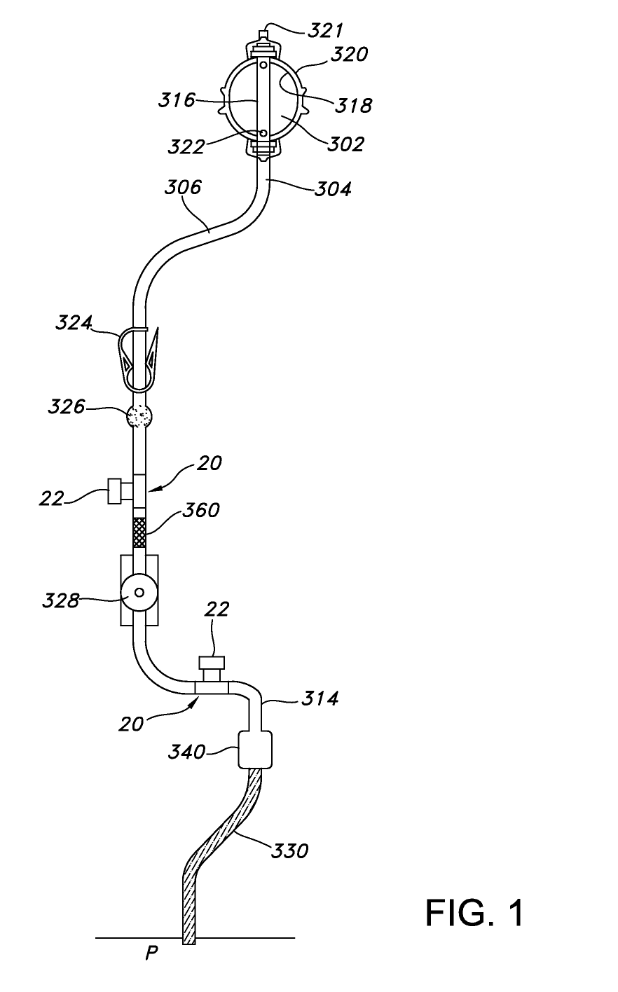

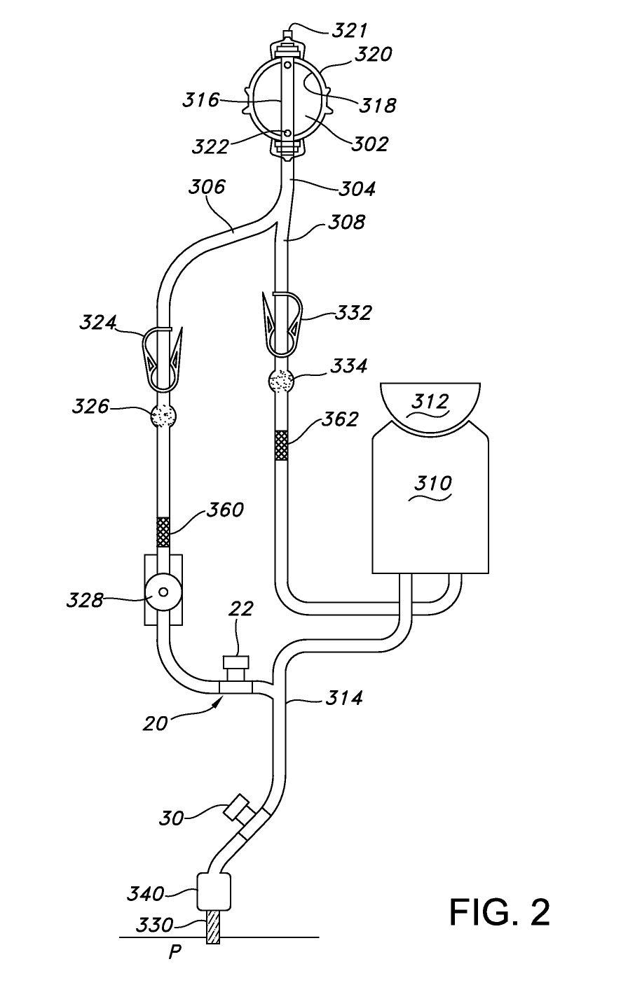

Referring to FIGS. 1 and 2, the present invention encompasses a device 300 for dispensing fluid to a patient and indicating a flow condition of the fluid through the device. The device 300 includes a reservoir 302 that serves as a pressurized fluid source or pump that holds medicinal fluid, such as local anesthetics (referred to hereinafter as a “pump”) and that is configured to provide a source of fluid under pressure. The pump 302 forces the medicinal fluid through a conduit 304. The conduit 304 forms a continuous flow path 306 for delivery into a wound site nerve bundle or the blood stream of a patient P.

In configurations that provide for bolus delivery as illustrated in FIG. 2, the conduit 304 splits into a continuous or primary flow path 306 and into a controlled bolus flow path 308 for delivery into a wound site nerve bundle or the blood stream of a patient P.

The pump 302 preferably accommodates about from 100 to 500 ml of fluid under 10-15 psi. The pump 302 has an inner core 316 surrounded by an elastomeric chamber 318 within a housing 320. The core 316 preferably has an inlet port 321 to fill the pump and an outlet port 322 in fluid communication with the tubing 304. The elastomeric chamber 318 is preferably constructed from a resilient material which may comprise a variety of elastomeric compositions, well known in the art, including vulcanized synthetic polyisoprenes, natural latex, natural rubber, synthetic rubber or silicone rubber. Fluid is held under pressure within the elastomeric chamber 318 and flows from the elastomeric chamber 318 through an outlet port 322 into the conduit 304 at a controlled and predictable rate. Alternatively, conduit 304 may be sized to serve as a flow restrictor. Exemplary pumps are described in U.S. Pat. No. 5,254,481 which is hereby incorporated by reference. A variety of other conventional pumps may be used, so long as they can impart the desired pressure on the fluid. For example, the pumps described in U.S. Pat. Nos. 5,080,652 and 5,105,983, which are hereby incorporated by reference may also be used, as well as other suitable electronic or mechanical pumps offered by other manufacturers as will be understood by those of skill in the art.

An optional clamp 324 is positioned in the flow path 306 downstream from the conduit 304. The clamp 324 can compress the flow path 306 such that fluid flow from the pump 302 is occluded. Such occlusion is advantageous for the transportation and preparation of the fluid delivery device and method as described herein. An exemplary clamp 324 is also described in U.S. Pat. No. 6,350,253, which is hereby incorporated by reference. However, a variety of other conventional clamps known in the industry may be used to occlude the flow of fluid from the pump 302 through the flow path 306 such as compression clamps, C clamps, roller clamps, and the like.

An optional filter 326 downstream of the clamp 324 separates the fluid from contaminates and other undesired particles that may be found within the fluid. The filter 326 also preferably eliminates air from the fluid path 306. One such filter 326 is described in U.S. Pat. No. 6,350,253, which is hereby incorporated by reference. Other suitable filters recognized in the industry may be used to capture undesired particles and/or remove air from the system.

An optional flow regulator 328 is positioned in the continuous flow path 306. The flow regulator 328 sets the continuous and substantially constant flow rate of fluid from the pump 302 to the patient P via tubing 304. The flow rate may be adjusted to a rate within a range of from about 0.5 to about 14 cubic centimeters of fluid per hour. Desirably, the flow rate may be from about 0.5 to about 7 or from about 1 to about 12 cubic centimeters per hour. The flow regulator 328 may be manually adjustable, if desired, and provided with a dial, switch or lever with an adjustable flow rate control display of from about 1 to about 14 cubic centimeters per hour. For example, the flow rate may be from about 1 to about 7 or from about 2 to about 14 cubic centimeters of fluid per hour. Alternatively, a constant flow regulator (i.e., a regulator which is not adjustable) can be employed. For example, an optional first flow regulating orifice such as a first glass orifice tube 360 may be employed in the primary or continuous flow path 306 and/or an optional second flow regulating orifice such as a second glass orifice tube 362 may be employed in the bolus flow path 308 (See FIG. 2).

The particular arrangement of the clamp 324, filter 326 and flow regulator 328 (or glass tube 360) herein described is merely exemplary. These elements, if present, may be arranged in any order as will be easily understood by those skilled in the art. Desirably, a first glass tube 360 and a second glass tube 362 are located downstream of the respective filters 326 and 334.

The device 300 for dispensing fluid to a patient utilizes at least one composition pulse flow sensor assembly 20 including a composition pulse flow sensor 22 to indicate a flow condition. Desirably, one composition pulse flow sensor assembly 20 with its composition pulse flow sensor is located above the flow regulator 328 (or alternatively the glass tube 360) and one composition pulse flow sensor assembly 20 with its composition pulse flow sensor is located below the flow regulator 328 (or alternatively the glass tube 360). The composition pulse flow sensor 22 provides a signal when the flow condition of the fluid in the continuous flow path 306 has changed from a predetermined flow condition. Generally speaking, the flow rate in the continuous flow path can be associated with a fluid flow state such as, for example, a continuous and steady flow rate. For example, the composition pulse flow sensor may be configured to provide a signal that the flow rate of the fluid in the continuous flow path is less than the predetermined flow rate, within a range of predetermined flow rates, or greater than a predetermined flow rate.

When a composition pulse flow sensor assembly with its composition pulse flow sensor is located between a flow regulator and the pressurized source of fluid and the composition pulse flow sensor provides a signal that the flow rate of the fluid in the continuous flow path is less than the predetermined flow rate, such a signal indicates a flow state that is less than a continuous and substantially constant flow rate of fluid. For example, if an outlet of the pressurized source of fluid becomes clogged, if a filter or bubble trap becomes clogged, a clamp inadvertently becomes closed or is left closed, or if the continuous flow path becomes kinked, pinched or constricted, the flow rate of fluid in the continuous flow path downstream of the obstruction will fall. The composition pulse flow sensor in fluid communication with the continuous flow path responds to the reduction in flow rate and provides a signal that may be made visible and/or audible to a care provider or a patient. The signal is interpreted as a reduction in the flow of fluid below a predetermined continuous and substantially constant flow rate.

When a composition pulse flow sensor assembly 20 with its composition pulse flow sensor 22 is located downstream of the flow regulator 328 (or orifice 360) and the composition pulse flow sensor provides a signal that the flow rate of the fluid in the continuous flow path is less than a predetermined flow rate, such a signal indicates a flow state that is less than a continuous and substantially constant flow rate of fluid. For example, if an outlet of the tubing 304 or connection of the continuous flow path to a catheter 330 becomes clogged, if the catheter 330 itself becomes clogged or if the tubing, continuous flow path or catheter becomes kinked, pinched or constricted, the flow rate of fluid will fall in the portion of the continuous flow path downstream of the flow regulator but upstream of an obstruction. The composition pulse flow sensor in fluid communication with the continuous flow path responds to the decrease in flow rate and provides a signal that may be made visible and/or audible to a care provider or a patient. The signal is interpreted as a reduction in the flow of fluid below a predetermined continuous and substantially constant flow rate.

Referring to FIG. 2, a large volume bolus delivery system 310 accumulates a large quantity of fluid from the bolus flow path 308 leading from the reservoir 302, and holds the fluid under pressure until the bolus dose is triggered by a patient operable actuator 312 for release into the patient P. The large volume bolus delivery system 310 is configured to receive fluid the bolus delivery system being configured to elastically expand to pressurize fluid, store the pressurized fluid and dispense the pressurized fluid while avoiding bolus refill during bolus delivery or after bolus delivery but before it is enabled to elastically expand in a subsequent delivery cycle. The actuator 312 is configured such that it does not require effort to force the fluid out of the bolus reservoir and that when actuated by the patient; fluid is permitted to flow out of the bolus reservoir to the patient without further action by the patient. The large volume bolus delivery system 310 is desirably the PCA device as described at, for example, U.S. Pat. No. 6,936,035 for “Patient Controlled Drug Administration Device” issued Aug. 30, 2005 to Rake et al. and U.S. patent application Ser. No. 12/968,988 filed Dec. 15, 2010 for “Improved Large-Volume Bolus Patient Controlled Drug Administration Device” by Valle et al., the contents of each are incorporated herein by reference.

Downstream from large volume bolus delivery system 310, the continuous flow path 306 and the bolus dose flow path 308 converge into a single flow path 314 to the patient P. Still referring to FIG. 2, an optional clamp 332 and an optional filter 334 may be positioned in the flow path 308 downstream from the conduit 304. The clamp 332 can compress the flow path 308 such that fluid flow from the pump 302 is occluded. Such occlusion is advantageous for the transportation and preparation of the fluid delivery device and method as described herein.

The release-rate of the bolus dose to the patient P is controlled by the decompression of the elastomeric bolus reservoir 310, by the pressure gradient at the valve 312, and the diameter of the catheter 330. Advantageously, the patient P does not have to provide pressure to force fluid out of the large volume bolus delivery system 310 into the narrower bolus flow path 308. Rather, the patient P can turn the stopcock or release the push button to administer the bolus dose. If the patient P activates the bolus valve 312 prior to the time the bolus reservoir 310 has filled to its capacity, the patient P receives less than the full amount of the bolus dose. In effect, this prevents the patient P from self-administering more than the maximum desired amount of fluid per the time specified as a large volume bolus dose.

A composition pulse flow sensor assembly 20 with its composition pulse flow sensor 22 may be located downstream of the location where the continuous flow path 306 and the bolus dose flow path 308 converge into a single flow path 314 as illustrated in FIG. 2. In this location, the flow sensor provides a signal that the flow rate of the fluid in the single flow path 314 is less than a predetermined flow rate; such a signal indicates a flow state that is less than a continuous and substantially constant flow rate of fluid. For example, if an outlet of the tubing 340 or connection of the continuous flow path to a catheter 330 becomes clogged or if the catheter itself becomes clogged, kinked, pinched or constricted, the flow rate of fluid will fall in the portion of the single flow path 314 upstream of an obstruction. The composition pulse flow sensor in fluid communication with the single flow path 314 responds to the decrease in flow rate and provides a signal that may be made visible and/or audible to a care provider or a patient. The signal is interpreted as a reduction in the flow of fluid below a predetermined continuous and substantially constant flow rate.

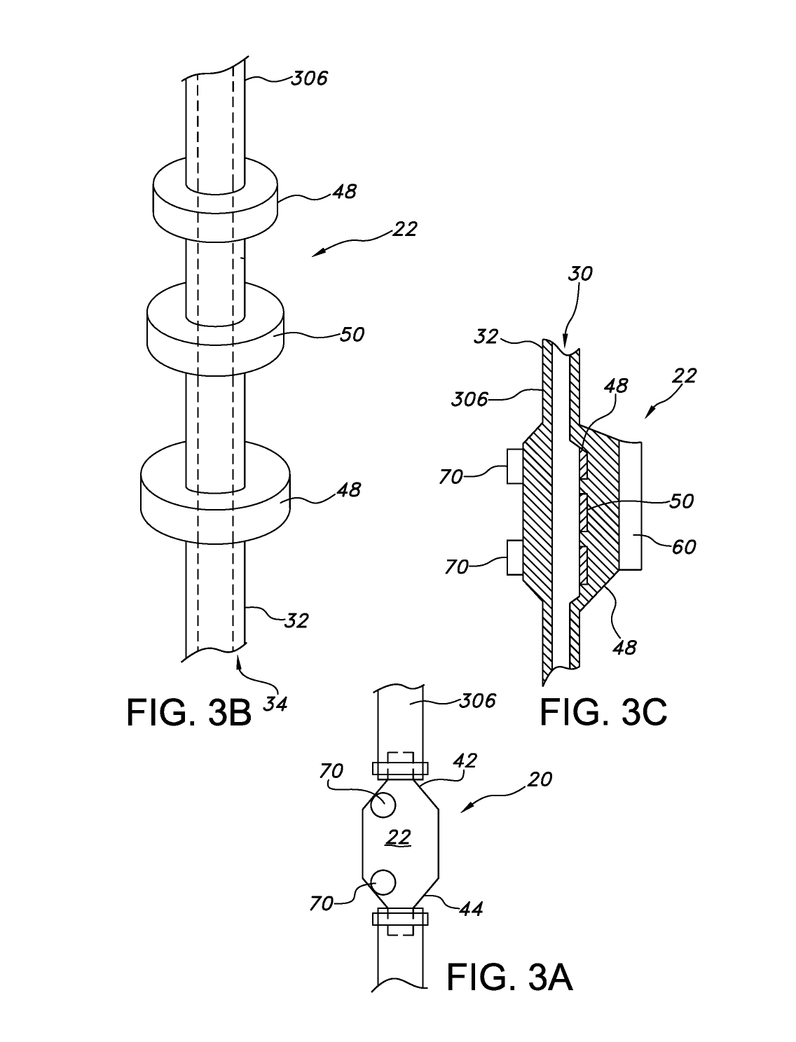

Turning now to FIGS. 3A though 3C, there is illustrated a composition pulse flow sensor assembly 20 or “detector assembly” incorporates a composition pulse flow sensor 22 that provides a signal when a flow condition in a continuous flow path 306 associated with the catheter 330 is different from a predetermined flow condition. The composition pulse flow sensor assembly 20 includes a continuous flow path 306 having walls 32 defining a lumen 34.

The composition pulse flow sensor 22 can be incorporated into a portion of the continuous flow path 306 in a variety of ways. For example, FIG. 3A illustrates the composition pulse flow sensor assembly 20 (with its composition pulse flow sensor) as a component having a first end having an inlet 42 for receiving fluid from the continuous flow path and a second end having an outlet 44 for returning fluid to the continuous flow path. The composition pulse flow sensor assembly can be joined, connected, or integrated in the continuous flow path by conventional techniques known to those of ordinary skill in the art.

Referring now to FIG. 3B, the composition pulse flow sensor 22 may include one or more sensors 48 (such as conductivity sensors) on either side of an electrode 50. These sensors 48 and the electrode may be annular such that they encircle the continuous fluid flow path. Alternatively and as illustrated in FIG. 3C, the one or more sensors 48 and the electrode 50 may be configured so they are flat films. For example, the one or more sensors 48 and the electrode 50 may be integrated in a semiconductor chip and the semiconductor chip mounted in a housing such that the sensor(s) and heating element are in fluid communication with the continuous flow path. A power source such as, for example, a battery 60 may be connected to the composition pulse flow sensor. One or more indicators 70 such as, for example, light-emitting diodes, speakers or the like may be incorporated in the composition pulse flow sensor assembly 20 (see FIG. 3A) or the composition pulse flow sensor 22 as shown in FIG. 3C.

During normal use of a composition pulse flow sensor assembly, pressurized fluid from the pump or reservoir 302 enters the conduit 304 and into the continuous or primary flow path 306. A regulator, which may be an adjustable regulator 328 or a non-adjustable flow regulating orifice 360, is used to control the flow of fluid. A fluid pressure exists within the continuous flow path and is normally associated with flow of fluid at a particular flow rate. A first composition pulse flow sensor assembly is located above the regulator. The detector assembly has a composition pulse flow sensor that responds to a predetermined flow rate or a range of flow rates in the continuous flow path. For example predetermined flow rate may be a range with a lower limit at the lower limit of the continuous and substantially constant flow rate of fluid (e.g., liquid drug) through of the continuous flow path that corresponds to a specific setting on a variable flow selector device and an upper limit that corresponds to a flow rates associated with a flow restrictor inserted in the continuous flow path. Alternatively and/or additionally, the predetermined flow rate or range of flow rates may be associated with the specific design of the infusion device, the liquid drug that is used with the device, or with other criteria.

An important feature of the present invention is that it provides a signal that the flow rate of a fluid in a continuous flow path is different from a predetermined flow rate or range of flow rates. Generally speaking, this can be accomplished by energizing a light source such as, for example, a light emitting diode. Alternatively and/or additionally, the signal may be an audible or tactile signal. For example, the device may energize a speaker to emit a “beep” or other sound. The device may emit a radio signal that is received by a secondary device that provides a visual, audible or tactile signal (e.g., a vibration).

The signal may be a “binary” signal. That is, the flow rate is either greater than (or equal to) the predetermined flow rate which provides one output from the composition pulse flow sensor or the flow rate is lower than the predetermined flow rate which provides a different output from the composition pulse flow sensor. This response may be much easier to interpret than the relative flow rates of devices that provide an uninterrupted reading or display of the different flow rates of a fluid in a continuous flow path. Of course, the composition pulse flow sensor may be utilized with a display or other signaling components that provide the relative flow rates through an uninterrupted reading or display of the different flow rates of a fluid in a continuous flow path.

In an aspect of the present invention, the composition pulse flow sensor assembly may include a composition pulse flow sensor that measures flow over time to provide information about a total volume of fluid (e.g., liquid drug) delivered past the flow detector. This information may be provided by a numeric display (e.g., numerals that correspond to units of fluid delivered) or it may be a signal such as a light or a beep that corresponds to a predetermined volume or a range of volumes. It is contemplated that one signal may be a binary signal providing information about the flow rate and a second, different signal may provide information about volume of fluid delivered.

An aspect of the present invention encompasses a composition pulse flow sensor assembly for indicating a fluid flow state in a medical device for dispensing a fluid under pressure to a patient through a continuous flow path at a continuous and substantially constant flow rate of fluid. The composition pulse flow sensor assembly includes a housing defining an inlet for receiving fluid from a continuous flow path and an outlet for returning fluid to the continuous flow path. The composition pulse flow sensor assembly also includes a composition pulse flow sensor contained in the housing. The composition pulse flow sensor of the assembly is in fluid communication with the continuous flow path. The composition pulse flow sensor provides a first signal when the flow rate of fluid in the flow path is at a predetermined flow rate and a second signal when the flow rate of fluid in the flow path is no longer at a predetermined flow rate. According to the invention, the second signal provides warning that indicates a flow state that is less than a continuous and substantially constant flow rate of fluid or that the flow rate is less than a predetermined flow rate.

The composition pulse flow sensor may provide such first and second signals with no signal of other states therebetween. That is, the composition pulse flow sensor may provide a signal of only two states of the continuous flow path—that it is at its predetermined flow state and that the continuous flow path is no longer at its predetermined flow state. The general structure of an exemplary composition pulse flow sensor assembly is as described above and is illustrated at, for example, in FIGS. 3A, 3B and 3C. For example, the composition pulse flow sensor assembly may include a composition pulse flow sensor composed of a channel in the housing for a fluid to be measured, a semiconductor chip arranged in the housing at the channel, the semiconductor chip having an integrated flow sensor. The integrated flow sensor is a composition pulse time-of-flight mass flow sensor composed of at least one electrode and at least one sensor. As noted above, exemplary flow detectors are described at, for example, U.S. Pat. No. 6,675,660; U.S. Pat. No. 7,225,683; and U.S. Pat. No. 7,703,336.

The present invention also encompasses a system for dispensing fluid to a patient and indicating a fluid flow condition. The system includes: a reservoir for providing a source of fluid under pressure; a continuous flow path in fluid communication with the source of fluid for providing a continuous and substantially constant flow rate of fluid from the source; and at least one composition pulse flow sensor in fluid communication with the continuous flow path, such that the at least one composition pulse flow sensor provides a signal when the flow rate of the fluid in the continuous flow path is different from a predetermined flow rate, thereby indicating a fluid flow condition.

The system may further include a flow regulator. A composition pulse flow sensor may be located between the flow regulator and the source of fluid such that the composition pulse flow sensor provides a signal when the flow rate of the fluid in the continuous flow path is less than the predetermined flow rate and such a signal indicates a flow state that is less than a continuous and substantially constant flow rate of fluid. Alternatively and/or additionally, the system may include a flow regulator and a composition pulse flow sensor may be located in a direction downstream of the flow regulator such that the composition pulse flow sensor provides a signal when the flow rate of the fluid in the continuous flow path is less than the predetermined flow rate and such a signal indicates a flow state that is less than a continuous and substantially constant flow rate of fluid.

While the present invention has been described in connection with certain preferred embodiments it is to be understood that the subject matter encompassed by way of the present invention is not to be limited to those specific embodiments. On the contrary, it is intended for the subject matter of the invention to include all alternatives, modifications and equivalents as can be included within the spirit and scope of the following claims.

We claim:

1. A device for dispensing fluid to a patient and indicating a fluid flow condition, the device comprising:
a reservoir configured to provide a source of fluid under pressure;
a continuous flow path from the source providing a continuous and substantially constant flow rate of fluid, and wherein the continuous flow path is in fluid communication with the source of fluid;
at least one composition pulse flow sensor in fluid communication with the continuous flow path, the at least one composition pulse flow sensor configured to indicate a fluid flow condition.

2. The device of claim 1, wherein the composition pulse flow sensor comprises a housing, a channel in the housing for a fluid to be measured, a semiconductor chip arranged in the housing at the channel, the semiconductor chip having an integrated flow sensor.

3. The device of claim 1, wherein the continuous flow path further includes a flow regulator which sets a flow rate through the continuous flow path into the patient.

4. The device of claim 3, wherein a composition pulse flow sensor is located between the flow regulator and the source of fluid.

5. The device of claim 3, wherein a composition pulse flow sensor is located in a direction downstream of the flow regulator.

6. The device of claim 2, wherein the integrated flow sensor is a composition pulse flow sensor comprising at least one electrode and at least one conductivity sensor.

7. The device of claim 2, wherein the integrated flow sensor is configured to determine flow velocity or the mass flow.

8. The device of claim 1, wherein the composition pulse flow sensor provides a visual signal to indicate a fluid flow condition.

9. The device of claim 2, wherein the composition pulse flow sensor provides an electronic signal to a display to indicate a fluid flow condition.

10. The device of claim 1, wherein the composition pulse flow sensor indicates a flow state that a continuous and substantially constant flow rate of fluid.

11. The device of claim 1, wherein the composition pulse flow sensor indicates a flow state that different from a predetermined flow state.

12. The device of claim 2, wherein the composition pulse flow sensor comprises:
a channel substrate;
at least one channel arranged in the channel substrate for receiving the fluid, wherein the channel is formed by at least one recess in the channel substrate;
a sensor substrate arranged at the channel substrate; and
a composition pulse flow sensor arranged on the sensor substrate, the flow sensor comprising at least one electrode and at least one sensor integrated on the sensor substrate,
wherein the channel substrate comprises a cover layer covering and closing the recess, and wherein the electrode and the sensor are in fluid contact with the cover layer and thereby with the channel, and
wherein the sensor substrate is arranged on an outer side of the cover layer.

13. The device of claim 1, further comprising:
a bolus flow path for the delivery of a bolus dose of fluid;
a large volume bolus delivery system in fluid communication with the bolus flow path and configured to receive fluid from the reservoir, the bolus delivery system being configured to elastically expand to pressurize fluid, store the pressurized fluid and dispense the pressurized fluid while avoiding bolus refill during bolus delivery or after bolus delivery but before it is enabled to elastically expand in a subsequent delivery cycle; and
a patient operable actuator to release fluid from the bolus delivery system, the actuator being configured such that it does not require effort to force the fluid out of the bolus reservoir and that when actuated by the patient, fluid is permitted to flow out of the bolus reservoir to the patient without further action by the patient.

14. A composition pulse flow sensor assembly for indicating a fluid flow state in a medical device for dispensing a fluid under pressure to a patient through a continuous flow path at a continuous and substantially constant flow rate of fluid, the composition pulse flow sensor assembly comprising:
a housing defining an inlet for receiving fluid from a continuous flow path and an outlet for returning fluid to the continuous flow path;
a composition pulse flow sensor contained in the housing, the composition pulse flow sensor providing a first signal when the flow rate of fluid in the flow path is at a predetermined flow rate and a second signal when the flow rate of fluid in the flow path is no longer at a predetermined flow rate, the composition pulse flow sensor being in fluid communication with the continuous flow path;
whereby the second signal provides warning that indicates a flow state that is less than a continuous and substantially constant flow rate of fluid.

15. The composition pulse flow sensor assembly of claim 14, wherein the composition pulse flow sensor comprises a channel in the housing for a fluid to be measured, a semiconductor chip arranged in the housing at the channel, the semiconductor chip having an integrated flow sensor.

16. The composition pulse flow sensor assembly of claim 14, wherein the integrated flow sensor is a composition pulse flow sensor comprising at least one electrode and at least one sensor.

17. The composition pulse flow sensor assembly of claim 14, wherein the composition pulse flow sensor comprises:
a channel substrate;
at least one channel arranged in the channel substrate for receiving the fluid, wherein the channel is formed by at least one recess in the channel substrate;
a sensor substrate arranged at the channel substrate; and
a composition flow sensor arranged on the sensor substrate, the flow sensor comprising at least one electrode and at least one sensor integrated on the sensor substrate,
wherein the channel substrate comprises a cover layer covering and closing the recess, and wherein the electrode and the sensor are in fluid contact with the cover layer and thereby with the channel, and
wherein the sensor substrate is arranged on an outer side of the cover layer.

18. A system for dispensing fluid to a patient and indicating a fluid flow condition, the system comprising:
a reservoir for providing a source of fluid under pressure;
a continuous flow path from the source providing a continuous and substantially constant flow rate of fluid, and wherein the continuous flow path is in fluid communication with the source of fluid;
at least one composition pulse flow sensor in fluid communication with the continuous flow path, the at least one composition pulse flow sensor providing a signal when the flow rate of the fluid in the continuous flow path is different from a predetermined flow rate, thereby indicating a fluid flow condition.

19. The system of claim 18, further comprising a flow regulator and wherein a composition pulse flow sensor is located between the flow regulator and the source of fluid such that the composition pulse flow sensor provides a signal when the flow rate of the fluid in the continuous flow path is less than the predetermined flow rate and such a signal indicates a flow state that is less than a continuous and substantially constant flow rate of fluid.

20. The system of claim 18, further comprising a flow regulator and wherein a composition pulse flow sensor is located in a direction downstream of the flow regulator such that the composition pulse flow sensor provides a signal when the flow rate of the fluid in the continuous flow path is less than the predetermined flow rate and such a signal indicates a flow state that is less than a continuous and substantially constant flow rate of fluid.
